SEQUENCE LISTING (8 sequences)

SEQ ID NO: 1 - DNA, 1308 bp, synthetic construct
  source 1..1308: mol_type = other DNA
atggccaagtacctggagctggaggagggcggcgtgatcatgcaggcgttctactgggacgtcccgagcggaggcatctggtgggacaccatccgccagaagatccccgagtggtacgacgccggcatctccgcgatctggataccgccagcttccaagggcatgtccgggggctactcgatgggctacgacccgtacgactacttcgacctcggcgagtactaccagaagggcacggtggagacgcgcttcgggtccaagcaggagctcatcaacatgatcaacacggcgcacgcctacggcatcaaggtcatcgcggacatcgtgatcaaccacagggccggcggcgacctggagtggaacccgttcgtcggcgactacacctggacggacttctccaaggtcgcctccggcaagtacaccgccaactacctcgacttccaccccaacgagctgcacgcgggcgactccggcacgttcggcggctacccggacatctgccacgacaagtcctgggaccagtactggctctgggcctcgcaggagtcctacgcggcctacctgcgctccatcggcatcgacgcgtggcgcttcgactacgtcaagggctacggggcctgggtggtcaaggactggctcaactggtggggcggctgggcggtgggcgagtactgggacaccaacgtcgacgcgctgctcaactgggcctactcctccggcgccaaggtgttcgacttccccctgtactacaagatggacgcggccttcgacaacaagaacatcccggcgctcgtcgaggccctgaagaacggcggcacggtggtctcccgcgacccgttcaaggccgtgaccttcgtcgccaaccacgacacggacatcatctggaacaagtacccggcgtacgccttcatcctcacctacgagggccagcccacgatcttctaccgcgactacgaggagtggctgaacaaggacaagctcaagaacctgatctggattcacgacaacctcgcgggcggctccactagtatcgtgtactacgactccgacgagatgatcttcgtccgcaacggctacggctccaagcccggcctgatcacgtacatcaacctgggctcctccaaggtgggccgctgggtgtacgtcccgaagttcgccggcgcgtgcatccacgagtacaccggcaacctcggcggctgggtggacaagtacgtgtactcctccggctgggtctacctggaggccccggcctacgaccccgccaacggccagtacggctactccgtgtggtcctactgcggcgtcggc

SEQ ID NO: 2 - protein, 436 aa, synthetic construct
MAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVG

SEQ ID NO: 3 - protein, 432 aa, synthetic construct
MKAILIPFLSLLIPLTPQSAFAQSEPELKLESVVIVSRHGVRAPTKATQLMQDVTPDAWPTWPVKLGELTPRGGELIAYLGHYWRQRLVADGLLPKCGCPQSGQVAIIADVDERTRKTGEAFAAGLAPDCAITVHTQADTSSPDPLFNPLKTGVCQLDNANVTDAILERAGGSIADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 4 - protein, 640 aa, synthetic construct
MTISRLSSVLFALALGQSALAAPQLSPRATTSLDAWLASETTVSLNGILDNIGASGAYAQSAKAGVVIASPSTSSPDYYYTWTRDSALTLKVLIDLFRNGNVDLQTVIEEYITAQAYLQTVSNPSGDLSSGAGLAEPKFNVDMSAYTGAWGRPQRDGPALRAIALIDFGNWLIDNGYSSYAVSNVWPIVRNDLSYVAQYWSQSGYDLWEEVNSMSFFTIANQHRALVEGSTFAGRVGASCSWCDSQAPQILCYMQNFWTGSYINANTGGGRSGKDANTVLASISTFDPEATCDDVTFQPCSSRALANHKVYTDSFRSVYSLDSGIAEGVAVAVGRYPEDSYYNGNPWFLTTLAAAEQLYDAIYQWNKIGSITITSTSLAFFNDVYSSAAVGTYASGSTAYTAIVSAVKTYADGYVSIVQAHAMTNGSLSEQFDKASGTQLSARDLTWSYAALLTANMRRNGIVPPSWGAASANSIPSSCSTGSATGTYSTPTGTSWPSTLTSGTAGTTTTSATTTTSTSVSKTTTTTTSTTSCTTPTSVAVTFDEIATTYYGENVYISGSISQLGSWDTSSAIALSASQYTSSNNLWFVTINLPAGTTFQYKYIRKESDGSIVWESDPNRSYTVPSGCGVSTATESDTWR

SEQ ID NO: 5 - protein, 422 aa, synthetic construct
MLKQFTKRLITLTSLLALVLVAPLASAGPLDGNSSDVMLQGFHWYSYQSFPWWGVIKNNAASIKADGFTMVWLPPPSDAASNEGYLPRRLELLDSKYGTRTDLVNALSALNANGVKPIADIVINHRVGTTGWADFTLPPWGSNAVCRGDEWSGATGNADTGDGFNAGRDIDHTQTFVQDGIVTWMNNSLKSVGFAGWRYDYVKGYSGSYVGSYNTRTTPYFSVGELWTDLDLNNPNPHRQLIMNWIDATGGRSAAFDFTTKGLLQQAVQYNEFWRLKDAAGAPAGAIGWWAAKSVTFIDNHDTGPSYPSGGQNHWPFPGDKILQGYAYILTHSGIPCVYWVHYKDWGQANTDAIKKLISIRKSKGITSTSSVSIQAADSSKYAAIITGNNGKVAVKIGFGAWSPPGTWTLATSGNNYAVWTQ

SEQ ID NO: 6 - protein, 461 aa, synthetic construct
MKLKYLALVLLAVASIGLLSTPVGAAKYSELEEGGVIMQAFYWDVPGGGIWWDTIRQKIPEWYDAGISAIWIPPASKGMGGGYSMGYDPYDFFDLGEYYQKGTVETRFGSKEELVNMINTAHSYGIKVIADIVINHRAGGDLEWNPFVNNYTWTDFSKVASGKYTANYLDFHPNEVKCCDEGTFGDFPDIAHEKSWDQYWLWASNESYAAYLRSIGIDAWRFDYVKGYGAWVVNDWLSWWGGWAVGEYWDTNVDALLNWAYDSGAKVFDFPLYYKMDEAFDNTNIPALVYALQNGGTVVSRDPFKAVTFVANHDTDIIWNKYPAYAFILTYEGQPVIFYRDYEEWLNKDKLNNLIWIHEHLAGGSTKILYYDNDELIFMREGYGSKPGLITYINLGNDWAERWVNVGSKFAGYTIHEYTGNLGGWVDRWVQYDGWVKLTAPPHDPANGYYGYSVWSYAGVG

SEQ ID NO: 7 - protein, 461 aa, synthetic construct; identical to SEQ ID NO: 6

SEQ ID NO: 8 - protein, 607 aa, synthetic construct
MKWTFSLLLLLSVFGQATHALTPAEWRSQSIYFLLTDRFGRTDNSTTAACDTTDRVYCGGSWQGIINHLDYIQGMGFTAIWITPVTGQFYENTGDGTSYHGYWQQDIYDLNYNYGTAQDLKNLASALHERGMYLMVDVVANHMGYDGAGNTVDYSVFNPFSSSSYFHPYCLISNYDNQTNVEDCWLGDTTVSLPDLDTTSTAVRDIWYDWVADLVANYSIDGLRVDTVKHVEKDFWPDYNSAAGVYCVGEVFSGDPAYTCPYQNYMDGVLNYPIYYQLLYAFESSSGSISDLYNMISSVASSCKDPTLLGNFIENHDNPRFASYTSDYSQAKNVITFIFLSDGIPIVYAGQEQHYSGGSDPANREATWLSGYSTSATLYTWIASTNQIRSLAISKDAGYVQAKNNPFYSDSNTIAMRKGTTAGAQVITVLSNKGASGSSYTLSLSGTGYSAGATLVETYTCTTVTVDSSGNLPVPMTSGLPRVFVPSSWVNGSALCNTECTAATSLPVLFEELVTTTYGENIYLSGSISQLGSWNTASAVALSASQYTSSNPKWYVSVTLPVGTSFQYKFIKKGSDGSVVWESDPNRSYTVPAGCEGATVTVADTWR

(19) United States
(12) Patent Application Publication
(10) Pub. No.: US 2025/0057096 A1
(43) Pub. Date: Feb. 20, 2025

(54) POLLEN-MEDIATED INDUSTRIAL TRAIT DELIVERY IN HYBRID F2 PROGENY SEED

(71) Applicant: INARI AGRICULTURE TECHNOLOGY, INC., Cambridge, MA (US)

(72) Inventors: Ponsi Trivisvavet, Lexington, MA (US); Claudia M. Nari, West Lafayette, IN (US); Ronald E. Wulfkuhle, Sioux Falls, SD (US)

(21) Appl. No.: 18/722,451

(22) PCT Filed: Dec. 29, 2022
(86) PCT No.: PCT/US2022/082525

§ 371 Date: Jun. 20, 2024

Related U.S. Application Data
(60) Provisional application No. 63/266,349, filed on Jan. 3, 2022.
(60) Provisional application No. 63/269,556, filed on Mar. 18, 2022.
(60) Provisional application No. 63/269,559, filed on Mar. 18, 2022.

(51) Int. Cl. A01H 1/00; C12N 9/32

(52) U.S. Cl. CPC A01H 1/102; C12N 9/2422; C12Y 302/01001

(57) ABSTRACT

A method for producing grain with improved fuel traits is presented, whereby the fuel trait is introduced directly into the grain through pollen applied from a donor source produced separate from the farmer's field, applied to the farmers field at the proper time, thereby eliminating multiple steps of trait introgression currently in practice for such grain production.

### CROSS REFERENCE TO RELATED APPLICATIONS

This application claims priority under 35 U.S.C. § 119 to provisional patent applications U.S. Ser. Nos. 63/269,559 and 63/269,556, both filed Mar. 18, 2022, and provisional patent application U.S. Ser. No. 63/266,349, filed Jan. 3, 2022. The provisional patent applications are herein incorporated by reference in their entirety, including without limitation, the specification, claims, and abstract, as well as any figures, tables, appendices, or drawings thereof.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Dec. 26, 2022, is named “P13791W000_SequenceListing.xml” and is 12,612 bytes in size.

### FIELD

The present disclosure relates generally to a method of improved delivery of a fuel trait to grain, through pollen, bypassing the need for trait introgression.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

### Development of Corn Hybrids

A single cross corn hybrid results from the cross of two inbred lines, each of which has a genotype that complements the genotype of the other. The hybrid progeny of the first generation is designated F₁. In the development of commercial hybrids in a corn plant-breeding program, only the F₁ hybrid plants are sought. Preferred F₁ hybrids are more vigorous than their inbred parents. This hybrid vigor, or heterosis, can be manifested in many polygenic traits, including increased vegetative growth and increased yield.

The development of a corn hybrid in a corn plant breeding program involves three steps: (1) the selection of plants from various germplasm pools for initial breeding crosses: (2) the selfing of the selected plants from the breeding crosses for several generations to produce a series of inbred lines, which, although different from each other, breed true and are highly uniform: and (3) crossing the selected inbred lines with different inbred lines to produce the hybrid progeny (F₁). During the inbreeding process in corn, the vigor of the lines decreases. Vigor is restored when two different inbred lines are crossed to produce the hybrid progeny (F₁). An important consequence of the homozygosity and homogeneity of the inbred lines is that the hybrid between a defined pair of inbreds will always be the same. Once the inbreds that give a superior hybrid have been identified, the hybrid seed can be reproduced indefinitely as long as the homogeneity of the inbred parents is maintained.

A single cross hybrid is produced when two inbred lines are crossed to produce the F₁ progeny. A double cross hybrid is produced from four inbred lines crossed in pairs (A×B and C×D) and then the two F₁ hybrids are crossed again (A×B)×(C×D). A three-way cross hybrid is produced from three inbred lines where two of the inbred lines are crossed (A×B) and then the resulting F₁ hybrid is crossed with the third inbred (A×B)×C. Much of the hybrid vigor exhibited by F₁ hybrids is lost in the next generation (F₂). Consequently, seed from hybrids is not used for planting stock.

Hybrid seed production requires elimination or inactivation of pollen produced by the female parent. Incomplete removal or inactivation of the pollen provides the potential for self-pollination.

Improvement of grain for fuel purposes is a key challenge in breeding and plant genetic improvement. One example of this is optimizing enzyme levels in grain used for fuel, such as amylase.

The possibility of using transgenic plants as a production system for valuable proteins has been proposed. Examples to date are the production of interferon in tobacco (Goodman, R. M., Knauf, V. C., Houck, C. M. and Comai, L. (1987) PCT/WO 87/00865), enkephalins in tobacco, Brassica nanus and Arabidopsis thaliana (Vandekerckhove, J., Van Damme, J., Van Lijsebettens, M., Botterman, J., DeBlock, M., DeClerq, A., Leemans, J., Van Montagu, M. and Krebbers, E. (1989) Bio/Technol. 7, 929), antibodies in tobacco (Hiatt, A., Cafferkey, R. and Boedish, K. (1990) Nature 342, 76) and human serum albumin in tobacco and potato (Sijmons, P.C., Dekker, B.M.M., Schrammeijer, B., Verwoerd, T. C., van den Elzen, P.J.M. and Hoekema, A. (1990) Bio/Technol. 8, 217).

Enzymes are used to process a variety of agricultural products such as wood, fruits and vegetables, starches, juices, and the like. Typically, processing enzymes are produced and recovered on an industrial scale from various sources, such as microbial fermentation (Bacillus α-amylase), or isolation from plants (coffee β-galactosidase or papain from plant parts). Enzyme preparations are used in different processing applications by mixing the enzyme and the substrate under the appropriate conditions of moisture, temperature, time, and mechanical mixing such that the enzymatic reaction is achieved in a commercially viable manner. One area where enzymes play an important role is in the area of corn milling.

Today corn is milled to obtain cornstarch and other corn-milling co-products such as corn gluten feed, corn gluten meal, and corn oil. The starch obtained from the process is often further processed into other products such as derivatized starches and sugars, or fermented to make a variety of products including alcohols or lactic acid.

Commercially, glucoamylases are used to further hydrolyze cornstarch, which has already been partially hydrolyzed with an alpha-amylase. The most widely utilized glucoamylase is produced from the fungus Aspergillus niger; one of the problems with the commercial use of this enzyme is its relatively low thermostability.

There is a need in the industry for new amylases, e.g., acid amylases, useful for various uses including commercial cornstarch liquefaction processes or improved manufacturing having new or improved performance characteristics over the industry standard enzymes, e.g., from Bacillus licheniformis. There is also an industry drive to identify amylases and glucoamylases capable of efficiently hydrolyzing granular starch (e.g., raw granular starch) at low temperatures without the need for a high temperature starch gelatinization step: the enzymes of the disclosure, e.g. amylases, glucoamylases and glucosidases, can be utilized to fulfill this need.

There is also a need for new amylases having utility in automatic dish wash (ADW)

products and laundry detergent. In ADW products, the amylase will function at pH 10-11 and at 45-60° C. in the presence of calcium chelators and oxidative conditions. For laundry, activity at pH 9-10 and 40° C. in the appropriate detergent matrix will be required. Amylases are also useful in textile desizing, brewing processes, starch modification in the paper and pulp industry and other processes described in the art.

It will be appreciated that an economical procedure for the production of enzymes such as amylase will be of significant benefit to, inter alia, the biofuels industry.

Thus, there exists a need in the art a novel system for delivering improved industrial traits to grain in a more efficient manner.

### GLOSSARY

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the phrase “genetically modified locus” can refer either to a transgene, an endogenous genetic locus which has been subjected to gene editing, or a transgene which has been subjected to gene editing. Genetically modified loci, including genetically modified loci of maize event 3272, and methods of gene editing are disclosed in WO2022/026375, WO2022/026379, WO 2022/026390, WO2022/026395, and WO2022/026403, each of which is incorporated herein by reference in its entirety.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context. The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

As used herein, the terms “include,” “includes,” and “including” are to be construed as at least having the features to which they refer while not excluding any additional unspecified features.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows steps in a current system for delivering a fuel trait into resultant grain.

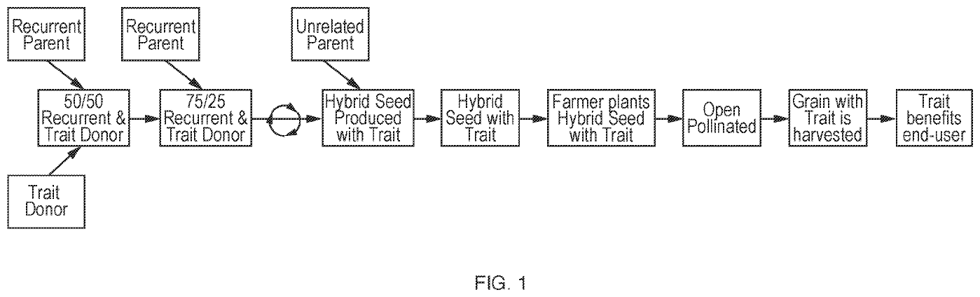

FIG. 2 shows the steps of an embodiment of the instant disclosure for delivering a fuel trait into resultant grain.

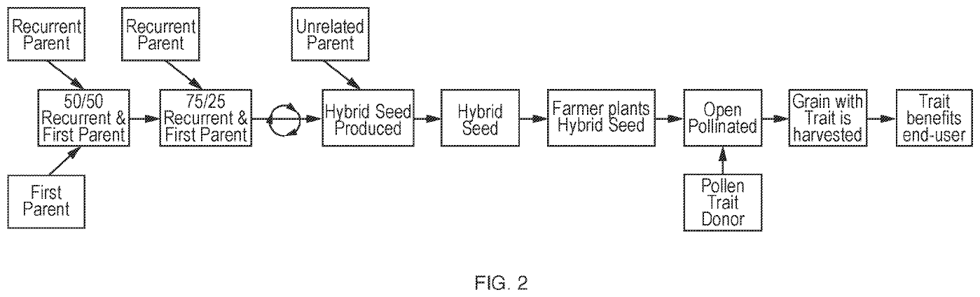

An artisan of ordinary skill need not view, within isolated figure(s), the near infinite number of distinct permutations of features described in the following detailed description to facilitate an understanding of the present disclosure.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art. When a male and female cross in corn, there is an effect of pollen on the biochemical properties of the next generation, the F2 seed/grain. This effect is called the xenia effect. The xenia effect is when pollen causes certain characteristics contributed by the genetics of the pollen to be expressed in the developing seed. These can be biochemical or morphological or both. The xenia effect is described in detail in Suaib Suaib, Sarawa Mamma, Tresjia Corina Rakian and Darwis Suleman, 2020. Xenia and Metaxenia in Maize Hybrid Varieties as a Consequence of Paternal Pollen Effect. Journal of Agronomy, 19:24-30, doi: 10.3923/ja.2020.24.30, incorporated herein by reference.

The xenia effect has been used in commercial corn production. It is the mechanism for which the high oil corn system known as TopCross Blend® utilized. In TopCross a blend of “pollinator” seeds (10-20% of seeds) and male sterile “grain” seeds (80-90% of seeds) were planted as a blend with the intent that the pollinator would pollinate the grain seeds and cause them to express a higher oil content. However, no commercial corn production system has taken advantage of this process for the transfer of GMO or gene edited traits and certainly not by pollination from pollen produced outside of the field.

Below, the process is described as specifically related to a trait, such as alpha amylase expression in maize. Herein, modified alpha amylase expression will be referred to as one non-limiting example of a “Trait”. The present disclosure relates to utilization of pollen from to a self-processing transgenic corn (Zea mays) plant that has incorporated into its genome a synthetic α-amylase gene (797GL3), encoding a thermostable 797GL3 α-amylase capable of processing starch in plants. Upon expression and activation of the α-amylase, the plant or plant part processes the substrate upon which the α-amylase acts. This “self-processing” results in significant improvement in making starch available for fermentation. Thus, methods which employ such plants and plant parts can eliminate the need to mill or otherwise physically disrupt the integrity of plant parts prior to recovery of starch-derived products. The transgenic corn event also has incorporated in its genome a manA gene, hereinafter called the pmi gene, encoding a phosphomannose isomerase enzyme (PMI), useful as a selectable marker, which allows the plant to utilize mannose as a carbon source. Other modifications that result in biochemical changes to the seed are other enzyme modifications, enhancements of amino acids, enhancements of proteins, oils and starches and would also be considered a Trait in this discussion. Non-limiting examples of Traits include those provided herein in Table 1.

Other uses will be apparent to those of skill in the art based upon the teachings provided herein, including those discussed above.

In the current production system, a farmer plants hybrid seed that has been introgressed by the seed company to contain the Trait. For introgression, a breeder would take the trait and back cross it from a donor parent into a recurrent parent and then the resulting seed crossed against the recurrent parent for successive generations until a target level of genetic purity is achieved, normally greater than 96% depending on the trait. To help prevent adventitious presence by pollen spreading to non-target fields, a trait like alpha amylase would be put into the female that is used to produce the hybrid seed the farmer plants. Then when the farmer harvests the hybrid seed, it is expressing the Trait. The downsides of this system are 1) the time to introgress a portfolio of hybrids, and, 2) limited hybrid availability of hybrid seed with the Trait. The Trait containing hybrid seed is then either fed as livestock feed or blended at an ethanol plant to a rate of 5-15% of grain used by the ethanol plant.

Steps of the Current System
1. Donor line containing the Trait is crossed against the “original” line that are the genetics that are intended to be used in the final product, also referred to as the recurrent line.
2. The F1 of the first cross that contains the Trait is crossed against the recurrent line resulting in seeds that are roughly 75% recurrent and 25% donor genetics.
3. The offspring from that cross is crossed again to the recurrent line and this is repeated until the percent of genetics of the original recurrent line is increased. Each cross reduces by half the percent of genetics from the Donor line.
   Recurrent parent X Donor seed Produced 50% recurrent/50% donor
   Recurrent Parent X F1 seed produced 75% recurrent/25% donor
   Recurrent Parent X BC1 seed Produced 87.5% recurrent/12.5% donor
   Recurrent Parent X BC2 seed produced 93.7% recurrent/6.25% donor
   Recurrent parent X BC3 seed produced 97% recurrent/3% donor

Once recovery of recurrent genetics is considered sufficient, a final step would be selfing to fix the line.
4. Once the desired level of original line genetics are obtained and the % of plants with the Trait normally 96-99%, the line is selfed to achieve homogeneity for use in increasing seed production.
   a. Molecular markers can be used in steps 1-4 to accelerate the selection for original genetics and purity.
5. The Traited line is then crossed against an unrelated line to produce hybrid seed that is sold to the farmer.
6. The farmer plants the hybrid seed, which is open pollinated from plant to plant, but primarily from pollen within that field.
7. The harvested grain resembles the % Trait that was in the original hybrid seed.
8. That Traited grain is then delivered to its destination, in the case of an output trait to an ethanol plant, livestock producer or food processor.

### EMBODIMENTS

Various embodiments of the systems and methods provided herein are included in the following non-limiting list of embodiments.
1. A method of producing maize grain comprising contacting a pollen recipient plant with a pollen formulation and harvesting grain comprising at least one genetically modified locus which confers an improved grain fuel trait from the pollen recipient plant, wherein the pollen recipient plant lacks said genetically modified locus.
2. The method of embodiment 1, wherein the at least one genetically modified locus which confers an improved grain fuel trait is selected from the group consisting of starch digestibility, starch content, amylase content, glutelin content, kernel weight, and kernel hardness, optionally wherein the trait is conferred by a genetically modified locus expressing a protein set forth in Table 1, Table 2, or a variant thereof having at least 95%, 96%, 97%, 98%, or 99% sequence identity thereto.
3. The method of embodiment 1 or 2, wherein the at least one genetically modified locus encodes an amylase protein, optionally wherein the amylase protein comprises an amylase set forth in Table 1, SEQ ID NO: 2, 5, 6, 7, 8, or a variant thereof having at least 95%, 96%, 97%, 98%, or 99% sequence identity thereto.
4. The method of anyone of embodiments 1 to 3, wherein the maize pollen formulation is a blend of pollen donor sources.
5. The method of any one of embodiments 1 to 4, wherein the pollen is obtained from a pollen donor located more than at least 200, 400, 600, 800, or 1,000 meters from the pollen recipient.
6. The method of any one of embodiments 1 to 5, wherein the contacting is mechanically mediated.
7. The method of any one of embodiments 1 to 6, wherein the pollen recipient plant is an F1 hybrid.
8. The method of any one of embodiments 1 to 7, wherein said pollen is provided in a formulation adapted for storage and/or for fertilization of a maize pollen recipient plant.
9. The method of any one of embodiments 1 to 8, further comprising containing and/or labelling the harvested grain as a grain lot.
10. The method of any one of embodiments 1 to 9, further comprising processing the grain to provide a fuel source component.
11. A grain lot comprising harvested grain obtained by the method of any one of embodiments 1 to 10.
12. A grain lot comprising harvested grain comprising a maize pollen recipient plant genome and a paternal genome comprising at least one modified locus which confers an improved grain fuel trait.
13. The grain lot of embodiment 12, wherein the at least one modified locus which confers an improved grain fuel trait is selected from the group consisting of starch digestibility, starch content, amylase content, kernel weight, and kernel hardness, optionally wherein the trait is conferred by a genetically modified locus expressing a protein set forth in Table 1, Table 2 or a variant thereof having at least 95%, 96%, 97%, 98%, or 99% sequence identity thereto.
14. The grain lot of embodiment 12 or 13, wherein the at least one genetically modified locus encodes an amylase protein, optionally wherein the amylase protein comprises an amylase set forth in Table 1, SEQ ID NO: 2, 5, 6, 7, 8, or a variant thereof having at least 95%, 96%, 97%, 98%, or 99% sequence identity thereto.
15. A single grain of the grain lot of any one of embodiments 11 to 14.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

In an embodiment of the new system, the farmer would plant a hybrid of his choice. Then when the hybrid seed starts to mature and silks emerge, pollen would be applied from a donor source. The donor source of pollen is produced separate from the farmer's field. It is harvested and then applied to the farmer's field at the proper time. As the farmer's hybrids will be fertile, the pollinations will be a mix of the original hybrid and the applied pollen. For the Trait a blend is acceptable to provide the benefit to the ethanol or livestock feeder.

In the new system, steps 1-5 are eliminated.
1. The farmer plants the hybrid seed of his choice (normally containing GMOs).
2. Concurrent to the farmer planting hybrid seed, the Trait donor line is planted, also a GMO or gene edited plant.
3. When the silks emerge and pollen begins to shed in the open pollinated system, pollen is harvested from the Trait donor lines, transferred and applied to the farmers hybrid field.
4. The resulting grain will contain the processor benefit of the Trait donor lines for those biochemical or morphological traits that result from the xenia effect.
5. The grain is harvested containing seeds that are a natural blend of open pollinated hybrid and plants pollinated from the donor exogenous source.

TABLE 1
Examples of Fuel Traits that could be introduced via pollen.

Altered amylase expression, including but not limited to the heterologous α-amylase
expressed from corn event 3272, comprising a transgenic genotype that comprises a
797GL3 α-amylase gene and a pmi gene which confers on the plant the ability to hydrolyze
starch under high temperatures and the ability to utilize mannose as a carbon source,
respectively, to the 3272 corn event and progeny thereof, as taught in U.S. Pat. No.
8,093,453, incorporated herein by reference in its entirety. In certain embodiments, the
genetically modified locus of corn event 3272 is present in seed deposited at the ATCC
under accession No. PTA-9972 or progeny thereof, is an allelic variant thereof, or is
another variant thereof. Genetically modified loci of maize event 3272 are disclosed in
WO2022/026375, WO2022/026379, WO2022/026390, WO2022/026395, and
WO2022/026403, each of which is incorporated herein by reference in its entirety.
Altered amylase expression, including but not limited to the heterologous a-amylase
expressed from a corn event 3272, comprising a transgenic genotype that comprises a
797GL3 α-amylase gene, while lacking a functional pmi marker gene, including but not
limited to lack of a functional pmi coding sequence and associated promoter.
Other enzymes and proteins expressed in a plant including enzymes and proteins encoded
by transgenes or endogenous genes that could be enhanced via promoter fine tuning and/or
enhancer insertion. Examples include:
(i) Amylases and Glucoamylases (including those disclosed in Table 2, U.S. Pat. No. 10,100,299,
incorporated herein by reference in its entirety, SEQ ID NO: 2, 4-8, or a variant thereof
having at least 95%, 96%, 97%, 98%, or 99% sequence identity thereto).
(ii) Glycosidases (EC 3.2.1), generally, and in particular, a-amylase (EC 3.2.1.1),
b-amylase (EC 3.2.1.2), g-amylase (EC 3.2.1.3), pullulinase, EC 3.2.1.41;
(iii) a stearyl-ACP desaturase;
(iv) a fructosyltransferase;
(v) a levansucrase;
(vi) an invertase; or
(vii) a starch branching enzyme.
Protein level
Protein digestibility
Starch digestibility
Starch content
Glutelin content
C-zein and D-zein protein content
Kernel weight
Kernel hardness

Particular genes and proteins of interest that can be introduced via pollen to confer Feed or Industrial Traits (e.g., grain fuel traits) include those set forth above in Table 1 and below in Table 2. Genes and proteins of interest that can be used in the methods and grain provided herein include variants having at least 95%, 96%, 97%, 98%, or 99% sequence identity to SEQ ID NO: 1-8.

TABLE 2

| SEQ | | | |
|---|---|---|---|
| ID | | | |
| NO | Type | Description | Sequence |
| 1 | DNA | Synthetic gene | ATGGCCAAGTACCTGGAGCTGGAGGAGGGCGG |
| | | encoding | CGTGATCATGCAGGCGTTCTACTGGGACGTCCC |
| | | 797GL3 α- | GAGCGGAGGCATCTGGTGGGACACCATCCGCC |
| | | amylase (from | AGAAGATCCCCGAGTGGTACGACGCCGGCATCT |
| | | U.S. 7557262) | CCGCGATCTGGATACCGCCAGCTTCCAAGGGCA |
| | | | TGTCCGGGGGCTACTCGATGGGCTACGACCCGT |
| | | | ACGACTACTTCGACCTCGGCGAGTACTACCAGA |
| | | | AGGGCACGGTGGAGACGCGCTTCGGGTCCAAG |
| | | | CAGGAGCTCATCAACATGATCAACACGGCGCAC |
| | | | GCCTACGGCATCAAGGTCATCGCGGACATCGTG |
| | | | ATCAACCACAGGGCCGGCGGCGACCTGGAGTG |
| | | | GAACCCGTTCGTCGGCGACTACACCTGGACGGA |
| | | | CTTCTCCAAGGTCGCCTCCGGCAAGTACACCGC |
| | | | CAACTACCTCGACTTCCACCCCAACGAGCTGCA |
| | | | CGCGGGCGACTCCGGCACGTTCGGCGGCTACCC |
| | | | GGACATCTGCCACGACAAGTCCTGGGACCAGTA |
| | | | CTGGCTCTGGGCCTCGCAGGAGTCCTACGCGGC |
| | | | CTACCTGCGCTCCATCGGCATCGACGCGTGGCG |
| | | | CTTCGACTACGTCAAGGGCTACGGGGCCTGGGT |
| | | | GGTCAAGGACTGGCTCAACTGGTGGGGCGGCTG |
| | | | GGCGGTGGGCGAGTACTGGGACACCAACGTCG |
| | | | ACGCGCTGCTCAACTGGGCCTACTCCTCCGGCG |
| | | | CCAAGGTGTTCGACTTCCCCCTGTACTACAAGA |
| | | | TGGACGCGGCCTTCGACAACAAGAACATCCCGG |
| | | | CGCTCGTCGAGGCCCTGAAGAACGGCGGCACG |
| | | | GTGGTCTCCCGCGACCCGTTCAAGGCCGTGACC |
| | | | TTCGTCGCCAACCACGACACGGACATCATCTGG |
| | | | AACAAGTACCCGGCGTACGCCTTCATCCTCACC |
| | | | TACGAGGGCCAGCCCACGATCTTCTACCGCGAC |
| | | | TACGAGGAGTGGCTGAACAAGGACAAGCTCAA |
| | | | GAACCTGATCTGGATTCACGACAACCTCGCGGG |
| | | | CGGCTCCACTAGTATCGTGTACTACGACTCCGA |
| | | | CGAGATGATCTTCGTCCGCAACGGCTACGGCTC |
| | | | CAAGCCCGGCCTGATCACGTACATCAACCTGGG |
| | | | CTCCTCCAAGGTGGGCCGCTGGGTGTACGTCCC |
| | | | GAAGTTCGCCGGCGCGTGCATCCACGAGTACAC |
| | | | CGGCAACCTCGGCGGCTGGGTGGACAAGTACGT |
| | | | GTACTCCTCCGGCTGGGTCTACCTGGAGGCCCC |
| | | | GGCCTACGACCCCGCCAACGGCCAGTACGGCTA |
| | | | CTCCGTGTGGTCCTACTGCGGCGTCGGC |
| 2 | PRT | 797GL3 α- | MAKYLELEEGGVIMQAFYWDVPSGGIWWDTIRQ |
| | | amylase (from | KIPEWYDAGISAIWIPPASKGMSGGYSMGYDPYD |
| | | U.S. 7781201) | YFDLGEYYQKGTVETRFGSKQELINMINTAHAYG |
| | | | IKVIADIVINHRAGGDLEWNPFVGDYTWTDFSKV |
| | | | ASGKYTANYLDFHPNELHAGDSGTFGGYPDICHD |
| | | | KSWDQYWLWASQESYAAYLRSIGIDAWRFDYVK |
| | | | GYGAWVVKDWLNWWGGWAVGEYWDTNVDAL |
| | | | LNWAYSSGAKVFDFPLYYKMDAAFDNKNIPALV |
| | | | EALKNGGTVVSRDPFKAVTFVANHDTDIIWNKYP |
| | | | AYAFILTYEGQPTIFYRDYEEWLNKDKLKNLIWIH |
| | | | DNLAGGSTSIVYYDSDEMIFVRNGYGSKPGLITYI |
| | | | NLGSSKVGRWVYVPKFAGACIHEYTGNLGGWVD |
| | | | KYVYSSGWVYLEAPAYDPANGQYGYSVWSYCG |
| | | | VG |
| 3 | PRT | Phytase (from | MKAILIPFLSLLIPLTPQSAFAQSEPELKLESVVIVS |
| | | U.S. 10428340) | RHGVRAPTKATQLMQDVTPDAWPTWPVKLGELT |
| | | | PRGGELIAYLGHYWRQRLVADGLLPKCGCPQSGQ |
| | | | VAIIADVDERTRKTGEAFAAGLAPDCAITVHTQA |
| | | | DTSSPDPLFNPLKTGVCQLDNANVTDAILERAGGS |
| | | | IADFTGHYQTAFRELERVLNFPQSNLCLKREKQDE |
| | | | SCSLTQALPSELKVSADCVSLTGAVSLASMLTEIF |
| | | | LLQQAQGMPEPGWGRITDSHQWNTLLSLHNAQF |
| | | | DLLQRTPEVARSRATPLLDLIKTALTPHPPQKQAY |
| | | | GVTLPTSVLFIAGHDTNLANLGGALELNWTLPGQ |
| | | | PDNTPPGGELVFERWRRLSDNSQWIQVSLVFQTL |
| | | | QQMRDKTPLSLNTPPGEVKLTLAGCEERNAQGM |
| | | | CSLAGFTQIVNEARIPACSL |
| 4 | PRT | Glucoamylase | MTISRLSSVLFALALGQSALAAPQLSPRATTSLDA |
| | | | WLASETTVSLNGILDNIGASGAYAQSAKAGVVIA |
| | | | SPSTSSPDYYYTWTRDSALTLKVLIDLFRNGNVDL |
| | | | QTVIEEYITAQAYLQTVSNPSGDLSSGAGLAEPKF |
| | | | NVDMSAYTGAWGRPQRDGPALRAIALIDFGNWLI |
| | | | DNGYSSYAVSNVWPIVRNDLSYVAQYWSQSGYD |
| | | | LWEEVNSMSFFTIANQHRALVEGSTFAGRVGASC |
| | | | SWCDSQAPQILCYMQNFWTGSYINANTGGGRSG |
| | | | KDANTVLASISTFDPEATCDDVTFQPCSSRALANH |
| | | | KVYTDSFRSVYSLDSGIAEGVAVAVGRYPEDSYY |
| | | | NGNPWFLTTLAAAEQLYDAIYQWNKIGSITITSTS |
| | | | LAFFNDVYSSAAVGTYASGSTAYTAIVSAVKTYA |
| | | | DGYVSIVQAHAMTNGSLSEQFDKASGTQLSARDL |
| | | | TWSYAALLTANMRRNGIVPPSWGAASANSIPSSC |
| | | | STGSATGTYSTPTGTSWPSTLTSGTAGTTTTSATT |
| | | | TTSTSVSKTTTTTTSTTSCTTPTSVAVTFDEIATTY |
| | | | YGENVYISGSISQLGSWDTSSAIALSASQYTSSNNL |
| | | | WFVTINLPAGTTFQYKYIRKESDGSIVWESDPNRS |
| | | | YTVPSGCGVSTATESDTWR |
| 5 | PRT | α-amylase | MLKQFTKRLITLTSLLALVLVAPLASAGPLDGNSS |
| | | | DVMLQGFHWYSYQSFPWWGVIKNNAASIKADGF |
| | | | TMVWLPPPSDAASNEGYLPRRLELLDSKYGTRTD |
| | | | LVNALSALNANGVKPIADIVINHRVGTTGWADFT |
| | | | LPPWGSNAVCRGDEWSGATGNADTGDGFNAGR |
| | | | DIDHTQTFVQDGIVTWMNNSLKSVGFAGWRYDY |
| | | | VKGYSGSYVGSYNTRTTPYFSVGELWTDLDLNNP |
| | | | NPHRQLIMNWIDATGGRSAAFDFTTKGLLQQAVQ |
| | | | YNEFWRLKDAAGAPAGAIGWWAAKSVTFIDNHD |
| | | | TGPSYPSGGQNHWPFPGDKILQGYAYILTHSGIPC |
| | | | VYWVHYKDWGQANTDAIKKLISIRKSKGITSTSS |
| | | | VSIQAADSSKYAAIITGNNGKVAVKIGFGAWSPPG |
| | | | TWTLATSGNNYAVWTQ |
| 6 | PRT | α-amylase | MKLKYLALVLLAVASIGLLSTPVGAAKYSELEEG |
| | | | GVIMQAFYWDVPGGGIWWDTIRQKIPEWYDAGIS |
| | | | AIWIPPASKGMGGGYSMGYDPYDFFDLGEYYQK |
| | | | GTVETRFGSKEELVNMINTAHSYGIKVIADIVINH |
| | | | RAGGDLEWNPFVNNYTWTDFSKVASGKYTANYL |
| | | | DFHPNEVKCCDEGTFGDFPDIAHEKSWDQYWLW |
| | | | ASNESYAAYLRSIGIDAWRFDYVKGYGAWVVND |
| | | | WLSWWGGWAVGEYWDTNVDALLNWAYDSGA |
| | | | KVFDFPLYYKMDEAFDNTNIPALVYALQNGGTV |
| | | | VSRDPFKAVTFVANHDTDIIWNKYPAYAFILTYEG |
| | | | QPVIFYRDYEEWLNKDKLNNLIWIHEHLAGGSTKI |
| | | | LYYDNDELIFMREGYGSKPGLITYINLGNDWAER |
| | | | WVNVGSKFAGYTIHEYTGNLGGWVDRWVQYDG |
| | | | WVKLTAPPHDPANGYYGYSVWSYAGVG |
| 7 | PRT | α-amylase | MKLKYLALVLLAVASIGLLSTPVGAAKYSELEEG |
| | | | GVIMQAFYWDVPGGGIWWDTIRQKIPEWYDAGIS |
| | | | AIWIPPASKGMGGGYSMGYDPYDFFDLGEYYQK |
| | | | GTVETRFGSKEELVNMINTAHSYGIKVIADIVINH |
| | | | RAGGDLEWNPFVNNYTWTDFSKVASGKYTANYL |
| | | | DFHPNEVKCCDEGTFGDFPDIAHEKSWDQYWLW |
| | | | ASNESYAAYLRSIGIDAWRFDYVKGYGAWVVND |
| | | | WLSWWGGWAVGEYWDTNVDALLNWAYDSGA |
| | | | KVFDFPLYYKMDEAFDNTNIPALVYALQNGGTV |
| | | | VSRDPFKAVTFVANHDTDIIWNKYPAYAFILTYEG |
| | | | QPVIFYRDYEEWLNKDKLNNLIWIHEHLAGGSTKI |
| | | | LYYDNDELIFMREGYGSKPGLITYINLGNDWAER |
| | | | WVNVGSKFAGYTIHEYTGNLGGWVDRWVQYDG |
| | | | WVKLTAPPHDPANGYYGYSVWSYAGVG |
| 8 | PRT | α-amylase | MKWTFSLLLLLSVFGQATHALTPAEWRSQSIYFLL |
| | | | TDRFGRTDNSTTAACDTTDRVYCGGSWQGIINHL |
| | | | DYIQGMGFTAIWITPVTGQFYENTGDGTSYHGYW |
| | | | QQDIYDLNYNYGTAQDLKNLASALHERGMYLMV |
| | | | DVVANHMGYDGAGNTVDYSVFNPFSSSSYFHPY |
| | | | CLISNYDNQTNVEDCWLGDTTVSLPDLDTTSTAV |
| | | | RDIWYDWVADLVANYSIDGLRVDTVKHVEKDF |
| | | | WPDYNSAAGVYCVGEVFSGDPAYTCPYQNYMD |
| | | | GVLNYPIYYQLLYAFESSSGSISDLYNMISSVASSC |
| | | | KDPTLLGNFIENHDNPRFASYTSDYSQAKNVITFIF |
| | | | LSDGIPIVYAGQEQHYSGGSDPANREATWLSGYS |
| | | | TSATLYTWIASTNQIRSLAISKDAGYVQAKNNPFY |
| | | | SDSNTIAMRKGTTAGAQVITVLSNKGASGSSYTLS |
| | | | LSGTGYSAGATLVETYTCTTVTVDSSGNLPVPMT |
| | | | SGLPRVFVPSSWVNGSALCNTECTAATSLPVLFEE |
| | | | LVTTTYGENIYLSGSISQLGSWNTASAVALSASQY |
| | | | TSSNPKWYVSVTLPVGTSFQYKFIKKGSDGSVVW |
| | | | ESDPNRSYTVPAGCEGATVTVADTWR |

### EXAMPLES

The following examples are prophetic.

### Example 1. Methods to Make 3 New Trait Combinations

Any corn hybrids can be pollinated with a donor exogenous to make 1) new GMO combinations, or 2) GMO x Edited combinations or 3) GMO by conventional combinations right in the farmers field rather than go through the lengthy costly process of introgressing the Trait into the seed the farmer plants.

### Example 2. New Method to Make Blended Trait Seed

Blended seed for the Trait is prepared in the farmers field by adding donor exogenous pollen to hybrid fields that are open pollinated which will result in a blend of Trait bearing and non-Trait bearing grains. For many Traits, it is not necessary to have the grain pure for the Trait, but blends are desired.

Example 3. Incorporation of fuel traits introduced into grain via pollen. Many traits impact grain quality for fuel purposes, including but not limited to carbohydrate and enzyme levels.

Examples of Traits that could be introduced as pollen and will express in the grain or are in theory would express are listed in Table 1 above.

Embodiments that would go across many different Traits that could be produced in this way.

From the foregoing, it can be seen that the present disclosure accomplishes at least all of the stated objectives.

The following table of reference characters and descriptors are not exhaustive, nor limiting, and include reasonable equivalents. If possible, elements identified by a reference character below and/or those elements which are near ubiquitous within the art can replace or supplement any element identified by another reference character.

What is claimed is:

1. A method of producing maize grain comprising contacting a pollen recipient plant with a pollen formulation and harvesting grain comprising at least one genetically modified locus which confers an improved grain fuel trait from the pollen recipient plant, wherein the pollen recipient plant lacks said genetically modified locus.

2. The method of claim 1, wherein the at least one genetically modified locus which confers an improved grain fuel trait is selected from the group consisting of starch digestibility, starch content, amylase content, glutelin content, kernel weight, and kernel hardness, optionally wherein the trait is conferred by a genetically modified locus expressing a protein set forth in Table 1 or 2.

3. The method of claim 1, wherein the at least one genetically modified locus encodes an amylase protein, optionally wherein the amylase protein comprises an amylase set forth in Table 1, SEQ ID NO: 2, 5, 6, 7, 8, or a variant thereof having at least 95%, 96%, 97%, 98%, or 99% sequence identity thereto.

4. The method of claim 1, wherein the maize pollen formulation is a blend of pollen donor sources.

5. The method of claim 1, wherein the pollen is obtained from a pollen donor located more than at least 200, 400, 600, 800, or 1,000 meters from the pollen recipient.

6. The method of claim 1, wherein the contacting is mechanically mediated.

7. The method of claim 1, wherein the pollen recipient plant is an F1 hybrid.

8. The method of claim 1, wherein said pollen is provided in a formulation adapted for storage and/or for fertilization of a maize pollen recipient plant.

9. The method of claim 1, further comprising containing and/or labelling the harvested grain as a grain lot.

10. The method of claim 1, further comprising processing the grain to provide a fuel source component.

11. A grain lot comprising harvested grain obtained by the method of any one of claims 1 to 10.

12. A grain lot comprising harvested grain comprising a maize pollen recipient plant genome and a paternal genome comprising at least one modified locus which confers an improved grain fuel trait.

13. The grain lot of claim 12, wherein the at least one modified locus which confers an improved grain fuel trait is selected from the group consisting of starch digestibility, starch content, amylase content, kernel weight, and kernel hardness.

14. The grain lot of claim 13, wherein the at least one genetically modified locus encodes an amylase protein, optionally wherein the amylase protein comprises an amylase set forth in Table 1, SEQ ID NO: 2, 5, 6, 7, 8, or a variant thereof having at least 95%, 96%, 97%, 98%, or 99% sequence identity thereto.

15. A single grain of the grain lot of claim 11 or 12.
